(19) United States
(12) Patent Application Publication
(10) Pub. No.: US 2024/0045011 A1
(43) Pub. Date: Feb. 8, 2024

(54) SYSTEM AND METHOD FOR RAPIDLY RECONSTRUCTING FUNCTIONAL MAGNETIC RESONANCE IMAGES

(71) Applicant: NOUS Imaging, Inc., St. Louis, MO (US)

(72) Inventors: Nico Dosenbach, St. Louis, MO (US); Ken Bruener, St. Louis, MO (US); Damien Fair, St. Louis, MO (US)

(21) Appl. No.: 18/266,236

(22) PCT Filed: Dec. 9, 2021
(86) PCT No.: PCT/US2021/062560

§ 371 Date: Jun. 8, 2023

Related U.S. Application Data
(60) Provisional application No. 63/123,302, filed on Dec. 9, 2020.

(51) Int. Cl. G01R 33/48; G01R 33/565; G01R 33/567; A61B 5/00

(52) U.S. Cl. CPC G01R 33/4806; G01R 33/56509; G01R 33/5676; A61B 5/746; A61B 5/055

(57) ABSTRACT

Systems and methods are provided for producing resting-state functional magnetic resonance imaging (rs-fMRI) images. The method may include receiving functional magnetic resonance imaging (fMRI) data acquired from a subject as the subject is subjected to at least one of performing a task or experiencing a stimulus and reconstructing the fMRI data acquired as the subject is subjected to at least one of performing a task or experiencing a stimulus using a resting-state fMRI (rs-fMRI) reconstruction process without accounting for the at least one of performing the task or experiencing the stimulus to generating rs-fMRI images. The method may also include displaying the rs-fMRI images and/or using the rs-fMRI images to determine motion of the subject during the acquisition of the fMRI data.

### CROSS-REFERENCE TO RELATED APPLICATIONS

This application is based on, is supported by, and incorporates herein by reference U.S. Provisional Application Ser. No. 63/123,302, filed Dec. 9, 2020.

### BACKGROUND

Patient motion of any kind (whether bulk rigid motion, or complex deformative motion) represents the greatest obstacle to preforming medical imaging. Some clinical applications require the precise acquisition of small signals with high spatial resolution, such that even small amounts of motion can substantially damage the clinical value of the information. For example, magnetic resonance imagining (MRI) of the brain represents a highly-valuable clinical application that is very susceptible to damaging the clinical value of the images with even small amounts of motion. For example, head motion damages the value of anatomical or structural (T1-weighted, T2-weighted, etc.) images and can be even more damaging to the clinical utility of so-called functional MRI data (fMRI). Even sub-millimeter head movements (e.g., micro-movements) may systematically alter structural and functional MRI data in some cases. Hence, much effort has been devoted towards developing post-acquisition methods for the removal of head motion distortions from MRI data.

Head movement from one MRI data frame to the next, rather than absolute movement away from the reference frame, is thought to induce the most significant MRI signal distortions. Motion-related distortions are strongly correlated with measures of framewise displacement (FD), which represent the sum of the absolute head movements in all six rigid body directions from frame to frame, as well as DVARS, which is the root mean square of the derivatives of the differentiated time courses of every voxel of an MRI image. Thus, measures such as FD and DVARS that capture the global effects of movement of the subject during MRI data acquisition, have been used to assess data quality in various post-hoc methods. For example, post-hoc frame censoring to remove all MRI data frames with FD values above a certain threshold (for example, excluding data frames with FD values >0.2 mm) has become a commonly used method for improving functional MRI data quality.

Though necessary for reducing artifacts, frame censoring comes at a steep price. For example, frame censoring can exclude 50% or more of the data in some studies. For example, so-called resting-state functional-connectivity MRI (rs-fcMRI) data can be particularly susecitpible to motion issues, because the studies, by definition, are extensive in length and focused on small signals elicited by the blood oxygen level dependent (BOLD) contrast mechanism. Because the accuracy of MRI measures improves as the number of frames increases, a minimum number of data frames may be required to obtain reliable data. If the number of frames remaining after censoring is too small, investigators may lose all data from a patient. In order to avoid this loss, clinicians typically collect additional “buffer” data, an expensive practice that, by itself, does not guarantee sufficient high-quality MRI data for a given participant. The overscanning required to remove motion-distorted data, while maintaining sample sizes adequate to achieve a desired data quality, has drastically increased the cost and duration of brain MRIs. Of course, in some ways the solution only exacerbates the problem. That is, the likelihood of patient motion increases with scan duration, so extending the scan to collect additional data only increases the likelihood of patient motion.

Recently developed structural MRI sequences with prospective motion correction use a similar approach to reduce the deleterious effects of head motion. These MRI sequences pair each structural data acquisition with a fast, low resolution, snap shot of the whole brain (e.g., echo-planar image, EPI) acquisition, which is then used as a marker or navigator for head motion. These motion-correcting structural sequences calculate relative motion between successive navigator images and use this information to mark the linked structural data frames for exclusion and reacquisition. In this manner, structural data frames are ‘censored,’ thereby increasing the duration and cost of structural MRIs.

These challenges with motion correction in the general context of fMRI are substantial. However, they are appreciably greater in some particular studies that, by necessity, are required to be extensive in length and are inherently intolerant of motion. For example, rs-fMRI, by definition, requires extended acquisitions that, likewise, extend the duration for scanning and, thereby, the opportunity for motion corruption of data and the costs for reacquisition. Furthermore, rs-fMRI is focused eliciting small signals from the brain, where fractions of a millimeter reflects to substantial variations in clinical information.

### SUMMARY OF THE DISCLOSURE

The present disclosure addresses the aforementioned drawbacks by providing systems and methods for controlling against the impact of motion on images produced using functional magnetic resonance imaging (fMRI). As one non-limiting example, as provided herein, a system and method for real-time motion identification may be used to improve and minimize acquisition times. Additionally or alternatively, a system and method for reconstruction of any form of fMRI data (i.e., BOLD-contrast data), whether acquired as task-based fMRI data or resting-state fMRI (rs-fMRI) data, may be reconstructed using an rs-fMRI reconstruction process, in accordance with the present disclosure.

In one configuration, a system is provided for performing a resting-state functional magnetic resonance image (rs-fMRI) reconstruction of functional magnetic resonance imaging (fMRI) dataset. The system includes a magnet system configured to generate a polarizing magnetic field about at least a portion of a subject, a magnetic gradient system including a plurality of magnetic gradient coils configured to apply at least one magnetic gradient field to the polarizing magnetic field, and a radio frequency (RF) system configured to apply an RF field to the subject and to receive magnetic resonance signals from the subject using a coil array. The system also includes a computer system programmed to control the magnetic gradient system and the RF system to an acquire fMRI dataset using at least one of a task-based fMRI data acquisition or an rs-fMRI data acquisition and, during the at least one of the task-based fMRI data acquisition or the rs-fMRI data acquisition, reconstruct the fMRI dataset using an rs-fMRI reconstruction process to generate at least one resting-state (rs) image. The computer system is further programmed to, during the at least one of the task-based fMRI data acquisition or the rs-fMRI data acquisition, compare the at least one rs image to a reference image to determine motion of the subject during the at least one of the task-based fMRI data acquisition or the rs-fMRI data acquisition and determine a displacement of the subject corresponding to the motion of the subject. The computer system is further configured to during the at least one of the task-based fMRI data acquisition or the rs-fMRI data acquisition, generate at least one of an alert or a real-time indication of the displacement that is communicated to an operator of the MRI system.

In one configuration, a computer-implemented method is provided for resting-state functional magnetic resonance imaging (rs-fMRI) reconstruction of functional magnetic resonance imaging (fMRI) dataset. The computer-implemented method includes receiving, using a computing device that includes at least one processor in communication with at least one memory device and that is in communication with a magnetic resonance imaging (MRI) system, an fMRI dataset from the MRI system while the MRI system is performing at least one of a task-based fMRI data acquisition or a rs-fMRI data acquisition. The computer-implemented method also includes performing an rs-fMRI reconstructing of the fMRI dataset, using the computing device, to generate rs-fMRI images and comparing, using the computing device and during the at least one of a task-based fMRI data acquisition or the rs-fMRI data acquisition, the rs-fMRI image to at least one reference image. The computer-implemented method further includes determining, using the computing device, motion of the subject using based on comparing the rs-fMRI image to the at least one reference image and communicating, using the computing device, an alert to an operator of the MRI system indicating motion detected during the at least one of the task-based fMRI data acquisition or the rs-fMRI data acquisition.

In another configuration, a system is provided for performing resting-state functional magnetic resonance imaging (rs-fMRI). The system includes a magnet system configured to generate a polarizing magnetic field about at least a portion of a subject, a magnetic gradient system including a plurality of magnetic gradient coils configured to apply at least one magnetic gradient field to the polarizing magnetic field and a radio frequency (RF) system configured to apply an RF field to the subject and to receive magnetic resonance signals from the subject using a coil array to form an rs-fMRI dataset according to an fMRI data acquisition. The system also includes a computer system programmed to receive the rs-fMRI dataset and compare the rs-fMRI dataset to reference dataset to determine motion of the subject, determine a displacement of the subject corresponding to the motion of the subject, and generate at least one of an alert or a real-time indication of the displacement that is communicated to an operator of the MRI system during the fMRI data acquisition

In yet another configuration, a method is provided for producing resting-state functional magnetic resonance imaging (rs-fMRI) images. The method includes receiving functional magnetic resonance imaging (fMRI) data acquired from a subject as the subject is subjected to at least one of performing a task or experiencing a stimulus. The method further includes reconstructing the fMRI data acquired as the subject is subjected to at least one of performing a task or experiencing a stimulus using a resting-state fMRI (rs-fMRI) reconstruction process without accounting for the at least one of performing the task or experiencing the stimulus to generating rs-fMRI images and displaying the rs-fMRI images.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention. Like reference numerals will be used to refer to like parts from Figure to Figure in the following description.

### BRIEF DESCRIPTION OF THE DRAWINGS

FIG. 1 is a flowchart of a non-limiting example of a method for processing a set of fMRI datasets in accordance with the present disclosure.

FIG. 2 is a flowchart illustrating a non-limiting example method for providing a sensory feedback to the operator of the MRI system and/or the patient within the MRI system during fMRI data acquisition.

FIG. 3 is a flowchart illustrating a non-limiting example method for removing artifacts associated with detected motion data using an Nth order filter.

FIG. 4 is a flowchart illustrating a non-limiting example adaptive filtering method.

FIG. 5 is a schematic of a system for performing magnetic resonance imaging in accordance with the present disclosure.

FIG. 6A is a block diagram of an example of a functional mapping-guided intervention targeting system.

FIG. 6B is a block diagram of components that can implement the functional mapping-guided intervention targeting system of FIG. 6A.

### DETAILED DESCRIPTION

Systems and methods are provided for processing fMRI data, such as resting state fMRI (rs-fMRI) data and/or task-based fMRI data, to reduce motion artifacts and scan times required to acquire all necessary data without motion corruption. Real-time monitoring and prediction of motion of a body part of a patient may include, but is not limited to, head motion during fMRI scanning. Methods, computer-readable storage devices, and systems are described for aligning fMRI data, such as datasets collected from an MRI scanner, to a reference dataset in order to monitor motion of a patient's body part during an fMRI scan. In various aspects, the reference dataset provides a common basis from which the displacement or motion of all datasets may be obtained and compared.

The fMRI data used in accordance with the present disclosure may include task-based fMRI data, rs-fMRI data, or a combination thereof, and may also be referred to as blood oxygenation level dependent (BOLD) data or BOLD activity data, when an activity or task is performed. Task-based fMRI includes MRI acquisitions where a subject performs a take or responds to stimulus while imaging is being performed. Resting-state fMRI is where the subject does not perform a task or respond to (or otherwise subjected to) stimulus while imaging is being performed. Instead, the subject lies in the MR scanner for a period of time while BOLD data is acquired. rs-fMRI demonstrates highly correlated low frequency (<0.1 Hz) changes in BOLD signals between different areas of the brain, which manifests the brain's functional connectivity. rs-fMRI presents unique challenges in that data must be acquired over long periods of time, and any motion by the subject may introduce detrimental errors or artifacts into the resulting images and thereby negate any diagnostic capability of the rs-fMRI scan.

Conventional fMRI image reconstruction involves acquiring fMRI data that includes both periods of a subject being at rest and a subject performing a task or experiencing stimuli. The acquired data is then processed with a traditional fMRI reconstruction or, as used herein, “fMRI reconstruction.” In an fMRI reconstruction, the fMRI is reconstructed based on periods of task/stimulus and periods without task/stimulus. Thus, the reconstruction is specifically designed around the distinction between periods of task or stimulus and periods without task or stimulus. For example, during the reconstruction, the data may be weighted to select the periods when a subject is performing a task and to mitigate any errors in the reconstructed images from periods when the subject is at rest.

In some aspects, the systems and methods in accordance with the present disclosure provide for reconstructing fMRI data of a subject that may include data acquired during performing a task or experiencing a stimulus (i.e., fMRI datasets or task-based fMRI datasets) using an rs-fMRI reconstruction process. In contrast to conventional approaches that weight task fMRI periods, the fMRI data may be reconstructed using only rs-fMRI processes. That is, the rs-fMRI process can be used in accordance with the present disclosure to disregard or without consideration to the distinction between task or stimulus. In this way, even when “traditional” or “task-based” fMRI data is acquired, a single, robust, set of images can be produced, even in the potential presence of motion during one portion of the acquisition. Furthermore, by disregarding distinctions or weightings for task and non-task or stimulus and non-stimulus data, images can be reconstructed rapidly. This rapid reconstruction without regard to distinctions or weightings for task and non-task or stimulus and non-stimulus data is referred to herein as rs-fMRI reconstruction because the reconstruction treats the data as though it was all acquired while the patient was at rest, whether this is true or not. By doing so, as stated, rapid reconstructions of images can be performed. This can be advantageous, for example, in a motion management process in accordance with the present disclosure. That is, these images reconstructed using a rs-fMRI reconstruction allows for reconstruction of images during the fMRI process (e.g., one not need wait for a completed acquisition over the whole process of tasks and stimulus or over an extended time period for a rs-fMRI acquisition) and these images produced during the fMRI data acquisition process can then be used to identify motion by the patient, as will be further described.

In various aspects, the systems and methods described herein may improve fMRI data quality and reduce costs associated with fMRI data acquisition. In one aspect, a method in accordance with the present disclosure is implemented in the form of software that calculates and displays data quality metrics and/or summary motion statistics in real time during an fMRI data acquisition (whether resting-state (rs-fMRI) or task-based fMRI). In a non-limiting example, the display may be in the form of a GUI generated during fMRI data acquisition and communicated to the clinician and/or the patient.

The systems and methods provided herein overcome one or more of at least several shortcomings of previous systems. To address the shortcomings associated with overscanning by previous systems to compensate for motion—distorted data, the systems and methods provided herein may provide real-time feedback to the scanner operator and/or the subject undergoing the scan. The operator may receive feedback in the form of a display quantifying the amount of motion a subject has experienced during a scan. Sensory feedback may be provided to a subject during the scan based on the data quality metrics and summary motion statistics calculated in real-time, thereby enabling the subject to monitor and adjust their movements accordingly (e.g., remain still) in response to the provided feedback. The systems and methods may include providing stimulus conditions, such as viewing a fixation crosshair or a movie clip, to simultaneously engage the subject while also providing real-time feedback to the subject.

For the purposes of this disclosure and accompanying claims, the term “real time” or related terms are used to refer to and defined a real-time performance of a system, which is understood as performance that is subject to operational deadlines from a given event to a system's response to that event. For example, a real-time extraction of data and/or displaying of such data based on empirically-acquired signals may be one triggered and/or executed simultaneously with and without interruption of a signal-acquisition procedure.

The shortcomings of conventional systems as described above may be addressed by enabling a scanner operator to continue each scan until the desired number of low-movement datasets have been acquired, in accordance with the present disclosure. Non-limiting examples of reaching the desired number of low-movement datasets include: (i) predicting the number of usable datasets that will be available at the end of the scan; (ii) predicting the amount of time a given subject will likely have to be scanned until the preset time-to-criterion (minutes of low-movement FD data) has been acquired; (iii) enabling for the selection and deselection of specific individual scans for inclusion in the actual and predicted amount of low-movement data, and the like.

Previously, motion estimates for brain MRIs were typically analyzed offline, either after data collection was completed for a given subject, or more commonly, in large batches after data collection for the whole cohort had been completed. Postponing head motion analyses is expensive and risky, especially when scanning a previously unstudied patient population and after making changes to the data collection protocol or personnel.

Real-time information about head motion can be used to reduce head motion in multiple different ways including, but not limited to: by influencing the behavior of MRI scanner operators, and by influencing MRI scanning subject behavior. Scanner operators may be alerted about any sudden or unusual changes in head movement and may be enabled to interrupt such scans to investigate if the subject has started moving more because they have grown uncomfortable and whether a bathroom break, blanket, repositioning or other intervention could make them feel more comfortable. In some aspects, the methods provided herein further include options for feeding information about head motion back to the subject, post-scan and/or in real time. The disclosed methods allow scanner operators to find the “sweet spot” that provides the required amount of low-movement data at the lowest cost. Following the methods, a scan could be stopped, the subject could be further instructed or reminded on ways to try remaining still, the scan could be re-acquired, and the like, to address motion.

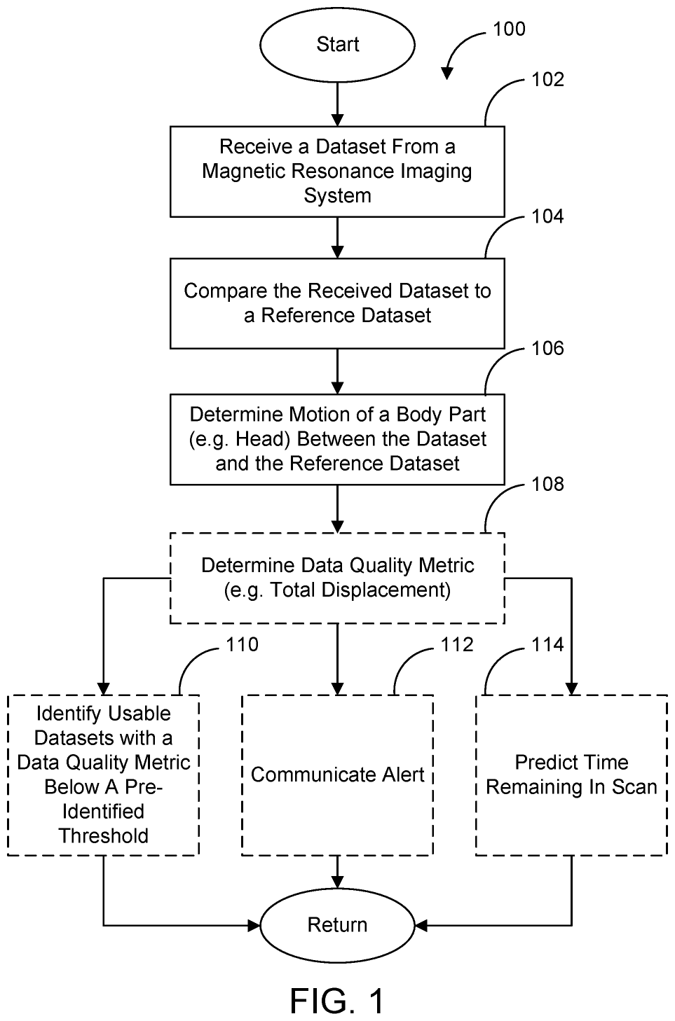

Referring to FIG. 1, a non-limiting example method 100 for processing a set of fMRI datasets to align the datasets to a reference dataset in a set to compensate for a subjects' movement, is shown. The method 100 includes receiving fMRI data at step 102 from a magnetic resonance imaging system in the form of an fMRI dataset or image/frame. The fMRI dataset may be received by a computing device from a magnetic resonance imaging system via a network or from a storage medium coupled to or in communication with the computing device. The fMRI dataset may be reconstructed using an rs-fMRI process, as described above in order to emphasize data acquired over resting-state periods rather than data acquired over periods of time when a subject was performing a task. Using a rs-fMRI reconstruction process also may prepare the data for motion correction, as described below.

The method 100 may also include comparing the dataset to the reference dataset at step 104. Each dataset or image may be aligned to the reference dataset through a series of rigid body transforms. Tᵢ, where i indexes the spatial registration of dataset i to a reference of dataset I, starting with the second dataset. Each transform may be calculated by minimizing the registration error to an absolute minimum or below a selected cutoff:

εᵢ= [symbol] (sIi(T(x))−I₁(\vec{x}))² [symbol] (1))

where I(x) is the image intensity at locus x and s is a scalar factor that compensates for fluctuations in mean signal intensity, spatially averaged over the whole brain, as depicted by the angle brackets. In some aspects, the datasets may be realigned using 4dfp cross_realign3d_4dfp algorithm (see Smyser, C. D. et al. 2010, Cerebral cortex 20, 2852-2862, (2010)) which is specifically incorporated herein by reference). Alternative alignment algorithms can also be utilized to align the datasets.

In various aspects, each transform may be represented by a combination of rotations and displacements as described by

$${T}_{i} = [ \begin{matrix}{R}_{i} & {d}_{i} \\ 0 & 1\end{matrix} ] \qquad ( 2 )$$

where Rᵢ represents the 3×3 matrix of rotations including the three elementary rotations at each of the three axes (see Example 1 below) and dᵢ represents the 3×1 column vector of displacements. Rᵢ may include the three elementary rotations at each of the three axes as expressed by: Rᵢ=R_{iα}R_{iβ}R_{iγ}, where

$${R}_{i α} = [ \begin{matrix}1 & 1 & 0 \\ 0 & cos ( {α}_{i} ) & - sin ( {α}_{i} ) \\ 0 & sin ( {α}_{i} ) & cos ( {α}_{i} )\end{matrix} ] \qquad ( 3 )$$

$${R}_{i β} = [ \begin{matrix}cos ( {β}_{i} ) & 0 & sin {β}_{i} \\ 0 & 1 & 0 \\ - sin {β}_{i} & 0 & cos {β}_{i}\end{matrix} ] \qquad ( 4 )$$

$${R}_{i γ} = [ \begin{matrix}cos ( {γ}_{i} ) & - sin ( {γ}_{i} ) & 0 \\ sin ( {γ}_{i} ) & cos ( {γ}_{i} ) & 0 \\ 0 & 0 & 1\end{matrix} ] \qquad ( 5 )$$

The method 100 may also include determining the relative motion of a body part between the dataset or image and the preceding dataset or image as indicated at step 106. In one non-limiting example, the method 100 may calculate the relative motion of the bodypart between a current image frame and a preceding image fame, at step 106. The relative motion of a body part (e.g., head motion) may be calculated from six alignment parameters, x, y, z, θ_{χ}, θ_{y}, and θ_{ζ}, where x, y, z, are translations in the three coordinate axis and θ_{χ}, θ_{y}, and θ_{ζ}, are rotations about those axis.

Optionally, the method 100 may also include determining a data quality metric, such as the total displacement at step 108 to generate multiple displacement vectors of, for example, head motion. In a non-limiting example, total displacement may be calculated by adding the absolute displacement of the body part (e.g., head) in six directions, thereby treating the body part as a rigid body. The head motion of the I^{th} dataset, such as the I^{th} frame, may be converted to a scalar quantity using the formula:

Displacement i=|Adᵢₓ|+|Ad_{iy}|+|Ad_{iz}|+|Acq|+|Δβ;|+|Δγ;| (6)

where Δdᵢₓ=d_{(i_1)x}−dᵢₓ; Δd_{iy}=d_{(i_1)y}−d_{iy}; Δd_{iz}=d_{(i_1)z}−d_{iz}; and so forth.

Rotational displacements |Δαᵢ|, |Δβᵢ|, and |Δγᵢ| may be converted from degrees to millimeters by computing displacement on the surface of a 3D volume representative of the body part being imaged. In a non-limiting example, if the head is imaged, the 3D volume selected to calculate displacement may be a sphere. Since each dataset is realigned to the reference dataset, displacement may be calculated by subtracting Displacement i−1 (for the previous dataset, which may correspond to a previous frame) from Displacement i (for the current dataset, which may correspond to a current frame).

In some aspects, the method 100 may further include excluding datasets with a cutoff above a pre-identified threshold of total displacement at step 110. Upon completion, the method 100 may return to the start for each subsequent dataset in the MRI scan. A display of the data quality metric may be performed at step 112, and a prediction of the time remaining in a scan may be performed at step 114.

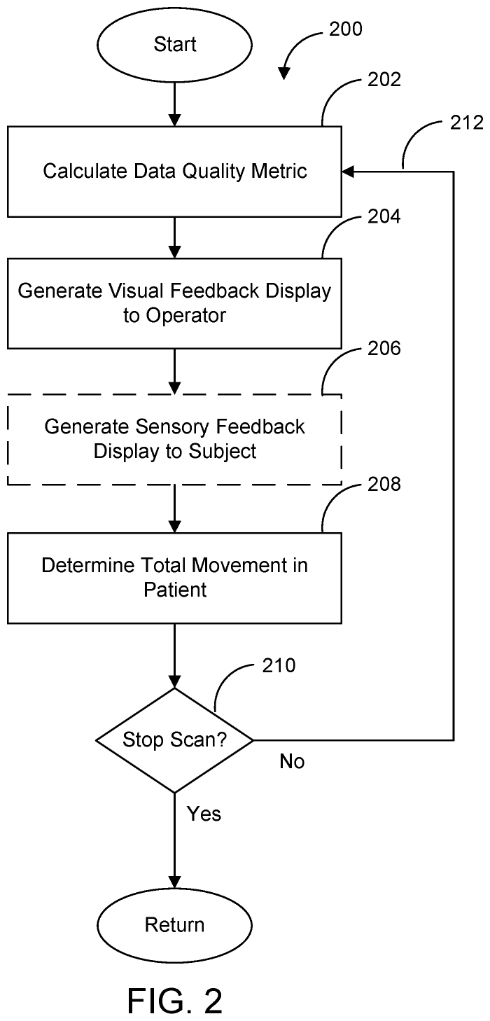

Referring to FIG. 2, a flow chart illustrating a non-limiting example method 200 for providing a sensory feedback to the operator of the MRI system and/or the patient within the MRI system during data acquisition is shown. The method 200 may include calculating a data quality metric at step 202 based on one or more components of movement determined for the patient in the MRI device during scanning. Any data quality metric may be calculated at 202 without limitation as described herein, including, but not limited to, any one or more of the displacement components as described above (e.g., framewise displacement, slice-by-slice displacement/motion), an overall displacement as described above, other data quality metrics including DVARS as described above, and any combination thereof.

The method 200 include generating a visual display in real-time to an operator of the MRI system at step 204 based on at least a portion of the data quality metric calculated at step 202. Non-limiting examples of suitable visual feedback displays include at least a portion of a GUI, a light bar, a video, an image, and the like. In various aspects, the visual feedback display for the operator of the MRI system may include visual elements including, but not limited to, one or more graphs displaying the data quality metrics for all datasets received in the scan, tables of summary statistics regarding the quality of the current and previous scans, graphical or tabular elements communicating the cumulative number of useable datasets obtained in the current scan, tabular or graphical elements communicating the amount of time remaining in the current scan and/or the predicted amount of time remaining in the current scan to obtain a predetermined number of useable scans, as described herein, and any combination thereof. In various aspects, the elements of the visual feedback display may be updated at any preselected rate up to a real-time rate of updating each display as each relevant quantity is calculated. The elements of the visual feedback display may be updated in response to a request from the operator of the MRI system, and the elements of the visual feedback display may dynamically updated in response to at least one of a plurality of factors including, but not limited to, significant increases in the monitored motion of the subject between datasets, cumulative motion, or any other suitable criteria.

The method 200 may optionally include generating a sensory feedback display at step 206 for the patient in the scanner during acquisition of fMRI data. As described in additional detail below, the sensory feedback display generated at step 206 may be updated at a wide variety of refresh rates ranging from a single update at the end of scanning to continuously updating in real time, based on at least one of a plurality of factors including, but not limited to the patient's age and condition.

In various aspects, the method 200 may further include determining the total movement of the patient at step 208 between the previous dataset and the current dataset in response to the sensory feedback display generated at step 206. In one aspect, the method 200 further includes evaluating at least one of a plurality of factors to determine whether the current fMRI scan should be terminated at step 210. In various aspects, the scan may be terminated in accordance with at least one of a plurality of termination criteria including, but not limited to, one of more movements of an unacceptably high magnitude, and unacceptably high number of relatively low magnitude movements, a determination that a suitable number of useable datasets were obtained, a prediction that a suitable number of useable datasets cannot be obtained in the time remaining in the scan, a prediction that a suitable number of useable datasets cannot be obtained within a reasonable cumulative scan time, and any combination thereof. If it is determined at step 210 to continue the scan, the method 200 may communicate at least one feedback signal 212 to be used in part to calculate the data quality metric at step 202 to start another iteration of the method 200 for a subsequent dataset.

In one aspect, the systems and methods provided herein may provide a visual feedback display to the subject undergoing the MRI scan. In this aspect, a characteristic of the visual feedback display may change to communicate the occurrence of movement of the subject based on the detected motion of the subject obtained using the method as described above. Any characteristic of one or more elements of a visual feedback display may be selected to vary in order to communicate the occurrence of movement including, but not limited to, a size, a shape, a color, a texture, a brightness, a focus, a position, a blinking rate, any other suitable characteristic of a visual element, and any combination thereof.

In another aspect, an auditory feedback display may be provided to the subject undergoing the MRI scan. A characteristic of the auditory feedback display may change to communicate the occurrence of movement of the subject based on the detected motion of the subject obtained using the method as described above. Any characteristic of one or more elements of an auditory visual feedback display may be selected to vary in order to communicate the occurrence of movement including, but not limited to, a pause in the playback of a musical selection, a resumption of playback of a musical selection, a verbal cue, a volume of a tone, a pitch of a tone, a duration of each tone in a series, a repeat rate of a series of tones, a steadiness or waver in a pitch or volume of a tone, any other suitable characteristic of an auditory feedback, and any combination thereof.

In various aspects, a characteristic of a sensory feedback display may vary based on a degree or magnitude of detected movement by the subject in the MRI scanner. In one aspect, the characteristic of the sensory feedback display may vary continuously in proportion to the degree of detected movement of the subject. In another aspect, the characteristic of the sensory feedback display may change within a discrete set of characteristics, in which each characteristic in the discrete set is configured to communicate the occurrence of one level of movement including, but not limited to, no movement, low movement, a medium or intermediate level of movement, and a high degree of movement.

In various other aspects, the sensory feedback display may vary in response to changes in a single component of movement such as a translation in a single x, y, or z direction or a rotation about a single x, y, or z direction, the sensory feedback display may vary in response to changes in a combination of two or more components of movement, or the sensory feedback display may vary in response to an overall movement metric such as displacement described above. In one aspect, a single characteristic of the sensory feedback display is varied to communicate the occurrence of movement to the subject. In another aspect, two or more characteristics of the sensory feedback are varied independently to communicate the occurrence of movement to the subject, in which each characteristic varies based on a subset of the components of movement. By way of non-limiting example, a sensory feedback display may include a first characteristic that varies based on movement of the subject in the x-direction, and a second characteristic that varies independently based on combined movement of the subject in the y-direction and z-direction. In a non-limiting example, the sensory feedback display may include color coded indications being displayed to the patient, such as using red to indicate motion has occurred in the x-direction, and green for motion in the y-direction, and blue for motion in the z-direction.

In various aspects, the frequency at which the characteristics of a sensory feedback display are updated may range from a single feedback display at the end of a scan to communicate whether or not sufficiently low movement was maintained during the scan to a frequency commensurate with the real-time frequency at which movement is monitored by the method, and at any intermediate frequency without limitation. In various aspects, the frequency at which the characteristics of a sensory feedback display are updated may be selected based on at least one characteristic of the subject to be imaged in the MRI scanner including but not limited to, age of the subject, a condition of the subject such as attention deficit disorder or a learning disability, and any other relevant characteristic of the subject without limitation. In various aspects, the method provides for feedback based on a motion value from a single dataset or a combination of motion values across multiple datasets. In various other aspects, the method provides for real-time feedback and time delayed feedback. By way of on-limiting example, if a high update frequency is used for a sensory feedback display for a very young child, the display may encourage the child to increase movement within the MRI scanner as a way of providing a more entertaining and dynamic sensory feedback experience. In various aspects, the frequency at which the characteristics of a sensory feedback display are updated may be specified to be a constant update rate throughout MRI scanning, or the update rate may dynamically vary based on an instantaneous and/or cumulative assessment of the motion of the subject.

In a non-limiting example of sensory feedback, a subject undergoing the fMRI scan may be instructed to view a fixation crosshair (e.g., a target). The crosshair may be color-coded based on the subject's detected movement (e.g., head motion), and the subject may be instructed to maintain the crosshair at a certain color (e.g., a first color) by remaining still during the scan. As a consequence of detected changes in the subject's movement, the crosshair may change to a second color (e.g., to represent medium movement) or a third color (e.g., to represent high movement), thereby enabling the subject to monitor and adjust his or her own movement during the scan.

In a non-limiting example of sensory feedback, a subject undergoing an MRI scan may be instructed to watch a movie clip. Based on the subject's level of movement (e.g. low movement, medium movement, high movement), a visual impediment on the movie clip may prevent the subject from viewing parts of the movie clip. For example, the subject may be instructed to remain still during the scan in order to watch an unobstructed view of the movie clip. Based on the subject's level of movement, the movie clip may be obstructed by a rectangular block of a certain size (e.g., a small yellow-colored rectangle for medium movement, and a large red-colored rectangular for high movement). Thus, the subject may be able to monitor and adjust his or her own movement during the scan based on the real-time visual feedback.

Fixed and adaptive feedback conditions may be provided for the real-time visual displays or sensory feedback. In one aspect for fixed feedback conditions, thresholds for low, medium, and high motions may be held constant for the duration of the fMRI scan. In another aspect for adaptive feedback conditions, thresholds for low, medium, and high motions may change and be replaced with stricter (e.g., lower) threshold values during the duration of the fMRI scan. With adaptive feedback conditions, the MRI scanner may adapt to the subject's ability to remain still, and, for example, increase the difficulty level of keeping the crosshair a first color or the movie clip visibly unobstructed.

In some aspects, changes in MRI acquisition procedures including, but not limited to, multiband imaging, enable improved temporal and spatial resolution relative to previous fMRI acquisition procedures. However, the improved temporal and spatial resolution may be accompanied by artifacts in motion estimates from post-acquisition dataset alignment procedures, thought to be caused primarily by chest motion during respiration. Without being limited to any particular theory, chest motion associated with respiration changes the static magnetic field (Bo) during MRI data acquisition, and such ‘tricks’ any dataset-to-dataset alignment procedure used in real-time motion monitoring into correcting a ‘head movement’ even in the absence of actual head movement. In one aspect, an optional band-stop (or notch) filter to remove respiration-related artifacts from motion estimates is provided, thereby enhancing the accuracy of real-time representations of motion.

A notch filter (e.g., band-stop filter) may be applied to motion measurements to remove artifacts from motion estimates caused by a subject's breathing. A subject's breathing may contaminate movement estimates in fMRI, and thereby distorts the quality of fMRI data obtained. Some aspects utilize a general notch filter to capture a large portion of a sample population's respiration peak with respect to power. A subject-specific filter based on filter parameters specific to a subject's respiratory belt data may be used.

The band-stop (e.g., notch filter) may be implemented to remove the spurious signal in the motion estimates that correspond to the aliased respiration rate. This filter may remove the undesired frequency components while leaving the other components unaffected. The notch filter may include design parameters of the central cutoff frequency and the bandwidth or range of frequencies that will be eliminated. To establish the parameters for the central cutoff frequency and the bandwidth, a distribution of respiration rates obtained from various subjects of fMRI during data acquisition may be analyzed, and a median of the distribution may be used as the cutoff frequency, and the quartiles 2 and 3 of the distribution may be used to determine bandwidths of the notch filter. Subsequent to establishing these parameters, an MR notch filter function may be used to design the notch filter. For a given sampling rate (1/TR), the respiratory rates may not be aliased. In other cases, when the combination of TR and respiration rate leads to aliasing, the aliased respiration rate may be used instead.

The designed filter may include a difference equation. When applied to a sequence representing a motion estimate, this difference equation may recursively weight the two previous samples to provide an instantaneous filtered signal. This procedure may start with the third sample, weight the two previous points, and continue until the last time-point is filtered. The filtered signal in such an implementation may have a phase delay with respect to the original signal. This phase delay may be compensated for by applying the filter twice, once forward and the second time backwards such that the opposite phase lags cancel out each other. Once the filter is applied to the entire sequence, the same filter (difference equation) may be reapplied backwards, with the last time-point of the forward-filtered sequence used as the first point for the backward application of the filter, and the recursive process may be continued until the first time-point of the forward-filtered sequence is filtered. In various aspects, the designed notch filters (general and subject-specific) may be applied to a sequence of motion estimates post-processing to improve data quality.

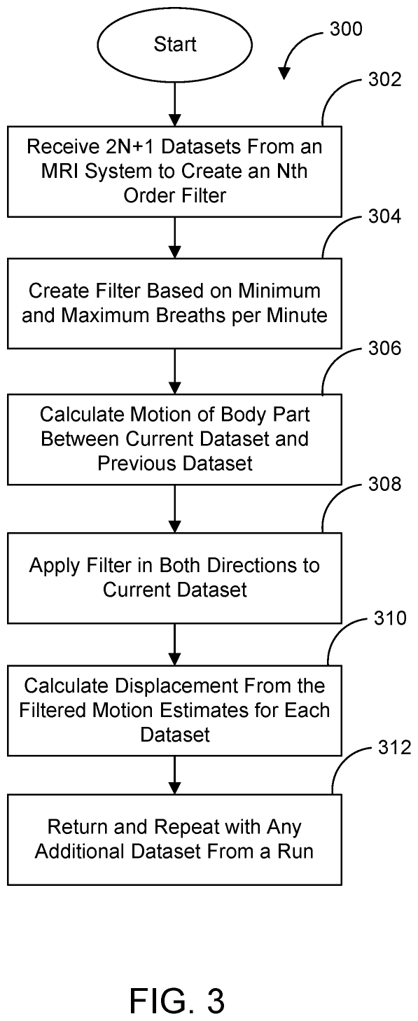

Referring to FIG. 3, a flow chart illustrating a non-limiting example method 300 for removing artifacts associated with detected motion data, such as respiration, using an Nth order filter is shown. In one aspect, the method 300 includes receiving 2N+1 datasets from the MRI system at step 302 that are used to create the Nth order filter. In addition to the 2N+1 datasets, the method 300 may further include creating the Nth order filter based on minimum and maximum respiratory frequencies at step 304. In various aspects, the subject's respiratory rate may be obtained using a variety of devices and methods including but not limited to, a respiratory monitor belt fitted to the subject, extracting respiratory frequency information from MRI signals obtained from the patient in the MRI scanner, and any other suitable method without limitation.

The method 300 may further include calculating a motion of a body part of the patient using the methods described herein at step 306. The method may further include applying the Nth order filter created at step 302 to the current dataset set in a forward and reverse direction with respect to data acquisition time at step 308. The Nth order filter in both directions may be used to eliminate a phase lag from the filtered data. Using the filtered motion estimates, a data quality metric including, but not limited to displacement may be calculated at step 310. If additional datasets are obtained at step 312, the method may replace the earliest dataset in the 2N+1 datasets received previously at step 302 with the dataset received at step 312 to initiate a subsequent iteration of the method 300.

The designed filter may also be applied in real time, since each instantaneous estimate of motion can be filtered out by weighting previous estimates following the notch filter's difference equation. In one aspect, the filter is run in pseudo-real time to minimize any resulting phase lag. In this aspect, once a specified number of samples are obtained, such as 5 samples in a non-limiting example, the filter could be applied twice and the best estimate would be the value corresponding to the third sample. This delayed signal will not have a phase delay. As each new sample is obtained, the filter can be applied twice to the entire sequence and the process can be repeated. Each time a new sample is measured, the filtered sequence will converge closer to the optimal output obtained when the filter is applied twice to the entire sequence. At the final dataset of a given run, the filtered sequence is then identical to the filtered sequence obtained during post-processing. Thus, the designed notch filters may be used in real-time to improve the accuracy of real-time estimates of motion using the motion prediction method described above.

In various aspects, adaptive filtering methods, including least squares adaptive filtering, may be applied in real-time to identify and remove signal content associated with undesired frequencies from subject movement data, such as cardiac and/or respiratory frequencies, from measured subject movement data including, but not limited to, displacement data, without concurrently introducing a phase lag to these data. In one aspect, a real-time adaptive filter may be used to remove respiratory-related artifacts from the fMRI data.

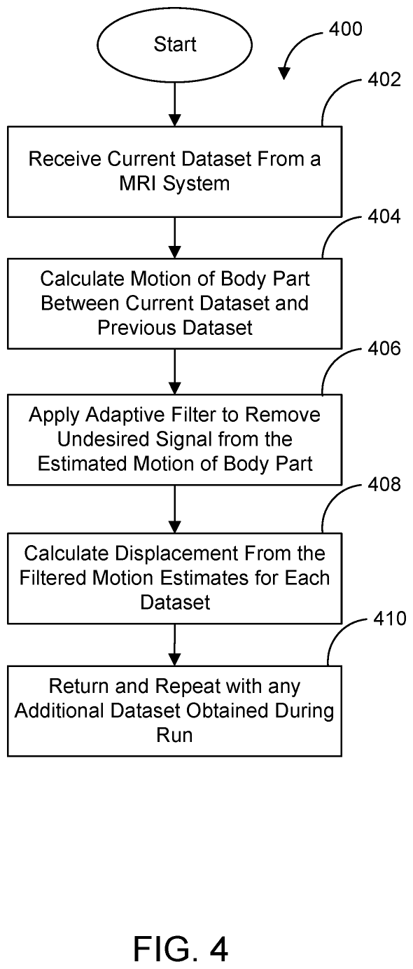

Referring to FIG. 4, a flowchart illustrating a non-limiting example adaptive filtering method 400 is shown. The method makes use of an unfiltered signal at step 402 including, but not limited to, displacement (such as, for example, framewise displacement (FD), slice-by-slice displacement, or the like) data derived from datasets obtained from the subject in the MRI scanner using the method described above, as well as a best estimate of the noise signal at step 404 to be eliminated using the adaptive filter at step 406. The adaptive filter method 400 may minimize in real time by gradient descent the contribution of the undesired signal into the measured signal, providing an optimal filtered sequence at step 408. The method may be repeated at step 410. Non-limiting examples of suitable noise signals to be input to the adaptive filter include real-time measurements of the respiration rate of the subject in the MRI scanner, the sum of multiple sinusoidal signals at different phases with frequencies corresponding to the respiration rate of the subject, and any other suitable estimate of the subject's respiration rate. In one aspect, the respiration rate of the participant could be measured while the T₁w or a previous sequence is acquired and used as the signal noise input.

In one aspect, the adaptive filter method 400 includes receiving a first estimation of head movement in each direction (i.e., x, y, z, θ_{χ}, θ_{y}, θ_{ζ}) as determined using the method described above. This first estimation of head movement includes both the real head movement (s) and the undesired artifact (n₀). These two signals (s) and (n₀) may be assumed to be independent and uncorrelated. An additional input may be used of a best estimation of the undesired artifact (n₁=n₀) received. If the undesired artifact no corresponds to respiration rate, this signal may be provided as a real time measurement of the respiration rate. In another aspect, if real time measurements of respiration are not available, a sinusoidal signal comprising a sum of a plurality of sinusoidal signals may be generated, in which the most likely respiration rate corresponds to the subject in the scanner. This error signal may be filtered out by the adaptive filter to generate an optimized estimate of the error signal (y(T)). In this aspect, the goal of the adaptive filter may be to maximize the correlation of the optimized estimate of the error signal (y(T)) and the measured estimation of head movement (d(T)). When the first dataset is used, the adaptive filter may have no effect on the signal (n₀). Also in this aspect, the optimized estimate of the error signal (y(T)) may be subtracted from the measured estimation of head movement (d(T)) to calculate the error signal (i.e. e(T)=s+n₀−y(T)). This error may be used as a feedback signal to modify the parameters of the adaptive filter to make the signal (y(t)) as correlated as possible to the measurement (d(T)). As the real head movement (s) and the real artifact (n₀) are uncorrelated, maximizing the correlation between no and d(T) may be driven by the match between no and no. Hence, subtracting those signals (d(T) and y(T)) removes the undesired artifact. In one aspect, an adaptive filter method may be implemented using well-established methods in which the parameters of a second order difference equation are optimized to maximize the estimation of the undesired artifact.

In various aspects, the methods in accordance with the present disclosure may be implemented by a system that includes an MRI system and one or more processors or computing devices. In various aspects, one or more operations described herein may be implemented by one or more processors having physical circuitry programmed to perform the operations. In various other aspects, one or more steps of the method may automatically be performed by one or more processors or computing devices. In various additional aspects, the various acts illustrated may be performed in the illustrated sequence, in other sequences, in parallel, or in some cases, may be omitted.

In some aspects, the above described methods and processes may be implemented using a computing system, including one or more computers. The methods and processes described herein may be implemented as a computer application, computer service, computer API, computer library, and/or other computer program product.

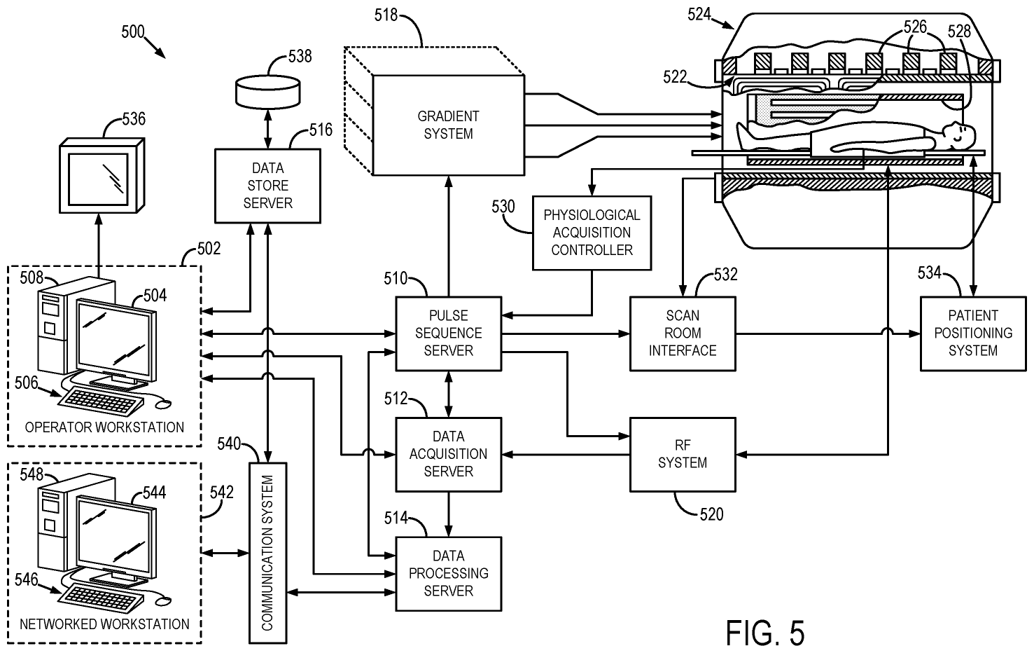

Referring to FIG. 5, an example of an MRI system 500 that can implement the methods described here is illustrated. The MRI system 500 includes an operator workstation 502 that may include a display 504, one or more input devices 506 (e.g., a keyboard, a mouse), and a processor 508. The processor 508 may include a commercially available programmable machine running a commercially available operating system. The operator workstation 502 provides an operator interface that facilitates entering scan parameters into the MRI system 500. The operator workstation 502 may be coupled to different servers, including, for example, a pulse sequence server 510, a data acquisition server 512, a data processing server 514, and a data store server 516. The operator workstation 502 and the servers 510, 512, 514, and 516 may be connected via a communication system 540, which may include wired or wireless network connections.

The pulse sequence server 510 functions in response to instructions provided by the operator workstation 502 to operate a gradient system 518 and a radiofrequency (“RF”) system 520. Gradient waveforms for performing a prescribed scan are produced and applied to the gradient system 518, which then excites gradient coils in an assembly 522 to produce the magnetic field gradients Gₓ, G_{y}, and G_{z} that are used for spatially encoding magnetic resonance signals. The gradient coil assembly 522 forms part of a magnet assembly 524 that includes a polarizing magnet 526 and a whole-body RF coil 528.

RF waveforms are applied by the RF system 520 to the RF coil 528, or a separate local coil to perform the prescribed magnetic resonance pulse sequence. Responsive magnetic resonance signals detected by the RF coil 528, or a separate local coil, are received by the RF system 520. The responsive magnetic resonance signals may be amplified, demodulated, filtered, and digitized under direction of commands produced by the pulse sequence server 510. The RF system 520 includes an RF transmitter for producing a wide variety of RF pulses used in MRI pulse sequences. The RF transmitter is responsive to the prescribed scan and direction from the pulse sequence server 510 to produce RF pulses of the desired frequency, phase, and pulse amplitude waveform. The generated RF pulses may be applied to the whole-body RF coil 528 or to one or more local coils or coil arrays.

The RF system 520 also includes one or more RF receiver channels. An RF receiver channel includes an RF preamplifier that amplifies the magnetic resonance signal received by the coil 528 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

M=\sqrt{I²+Q²} (7);

and the phase of the received magnetic resonance signal may also be determined according to the following relationship:

$$φ = {tan}^{- 1} ( \frac{Q}{I} ) . \qquad ( 8 )$$

The pulse sequence server 510 may receive patient data from a physiological acquisition controller 530. Byway of example, the physiological acquisition controller 530 may receive signals from a number of different sensors connected to the patient, including electrocardiograph (“ECG”) signals from electrodes, or respiratory signals from a respiratory bellows or other respiratory monitoring devices. These signals may be used by the pulse sequence server 510 to synchronize, or “gate,” the performance of the scan with the subject's heart beat or respiration.

The pulse sequence server 510 may also connect to a scan room interface circuit 532 that receives signals from various sensors associated with the condition of the patient and the magnet system. Through the scan room interface circuit 532, a patient positioning system 534 can receive commands to move the patient to desired positions during the scan.

The digitized magnetic resonance signal samples produced by the RF system 520 are received by the data acquisition server 512. The data acquisition server 512 operates in response to instructions downloaded from the operator workstation 502 to receive the real-time magnetic resonance data and provide buffer storage, so that data is not lost by data overrun. In some scans, the data acquisition server 512 passes the acquired magnetic resonance data to the data processor server 514. In scans that require information derived from acquired magnetic resonance data to control the further performance of the scan, the data acquisition server 512 may be programmed to produce such information and convey it to the pulse sequence server 510. For example, during pre-scans, magnetic resonance data may be acquired and used to calibrate the pulse sequence performed by the pulse sequence server 510. As another example, navigator signals may be acquired and used to adjust the operating parameters of the RF system 520 or the gradient system 518, or to control the view order in which k-space is sampled. In still another example, the data acquisition server 512 may also process magnetic resonance signals used to detect the arrival of a contrast agent in a magnetic resonance angiography (“MRA”) scan. For example, the data acquisition server 512 may acquire magnetic resonance data and processes it in real-time to produce information that is used to control the scan.

The data processing server 514 receives magnetic resonance data from the data acquisition server 512 and processes the magnetic resonance data in accordance with instructions provided by the operator workstation 502. Such processing may include, for example, reconstructing two-dimensional or three-dimensional images by performing a Fourier transformation of raw k-space data, performing other image reconstruction algorithms (e.g., iterative or backprojection reconstruction algorithms), applying filters to raw k-space data or to reconstructed images, generating functional magnetic resonance images, or calculating motion or flow images.

Images reconstructed by the data processing server 514 are conveyed back to the operator workstation 502 for storage. Real-time images may be stored in a data base memory cache, from which they may be output to operator display 502 or a display 536. Batch mode images or selected real time images may be stored in a host database on disc storage 538. When such images have been reconstructed and transferred to storage, the data processing server 514 may notify the data store server 516 on the operator workstation 502. The operator workstation 502 may be used by an operator to archive the images, produce films, or send the images via a network to other facilities.

The MRI system 500 may also include one or more networked workstations 542. For example, a networked workstation 542 may include a display 544, one or more input devices 546 (e.g., a keyboard, a mouse), and a processor 548. The networked workstation 542 may be located within the same facility as the operator workstation 502, or in a different facility, such as a different healthcare institution or clinic.

The networked workstation 542 may gain remote access to the data processing server 514 or data store server 516 via the communication system 540. Accordingly, multiple networked workstations 542 may have access to the data processing server 514 and the data store server 516. In this manner, magnetic resonance data, reconstructed images, or other data may be exchanged between the data processing server 514 or the data store server 516 and the networked workstations 542, such that the data or images may be remotely processed by a networked workstation 542.

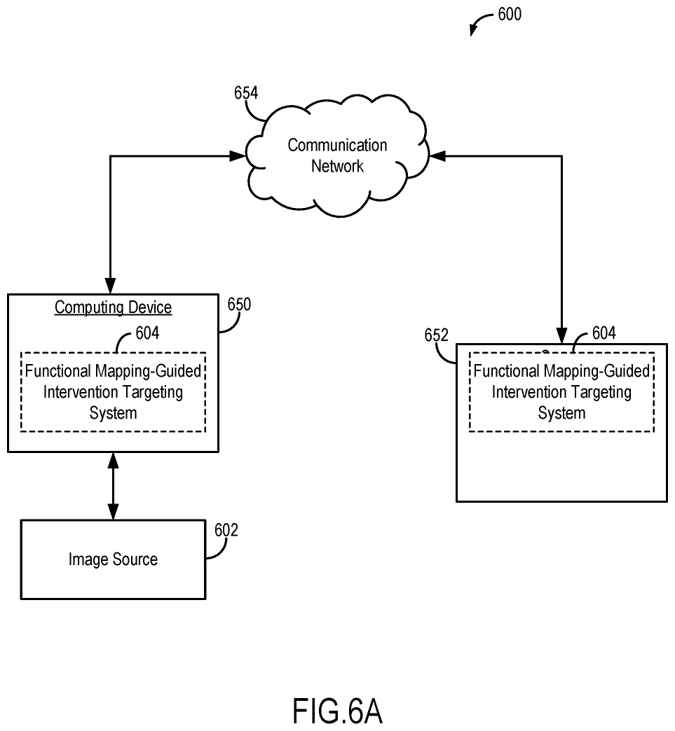

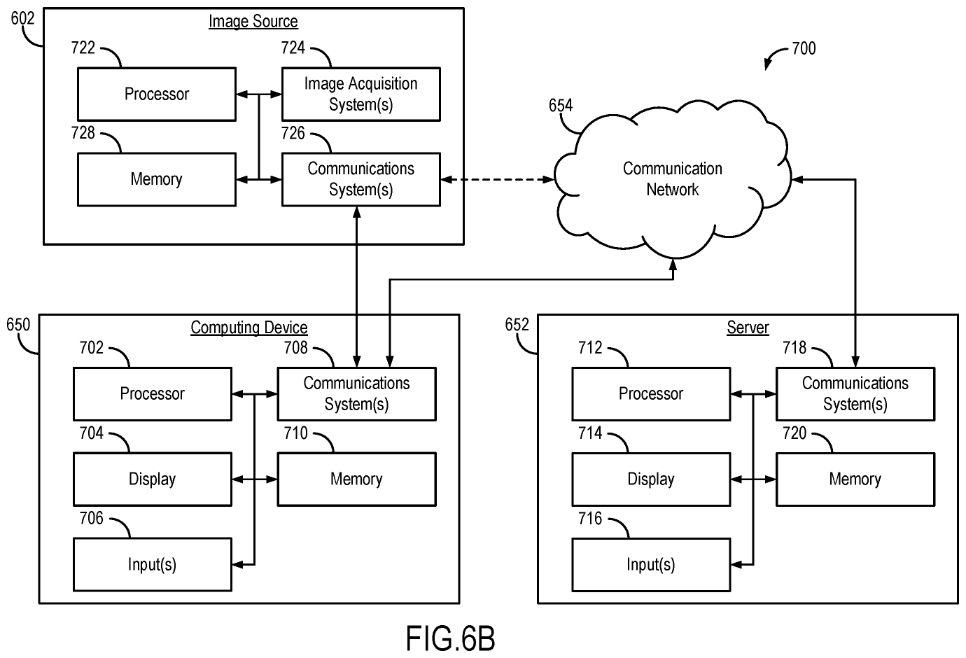

Referring now to FIG. 6A, an example of a system 600 for functional mapping-guided intervention targeting (e.g., determination of anatomical targets for brain stimulation) in accordance with some embodiments of the systems and methods described in the present disclosure is shown. As shown in FIG. 6A, a computing device 650 can receive one or more types of data (e.g., fMRI, task-based fMRI, and/or rs-fMRI data) from image source 602, which may be an MRI source. In some embodiments, computing device 650 can execute at least a portion of a functional mapping-guided intervention targeting system 604 to generate intervention targets from data received from the image source 602.

Additionally or alternatively, in some embodiments, the computing device 650 can communicate information about data received from the image source 602 to a server 652 over a communication network 654, which can execute at least a portion of the functional mapping-guided intervention targeting system 604. In such embodiments, the server 652 can return information to the computing device 650 (and/or any other suitable computing device) indicative of an output of the functional mapping-guided intervention targeting system 604.

In some embodiments, computing device 650 and/or server 652 can be any suitable computing device or combination of devices, such as a desktop computer, a laptop computer, a smartphone, a tablet computer, a wearable computer, a server computer, a virtual machine being executed by a physical computing device, and so on. The computing device 650 and/or server 652 can also reconstruct images from the data.

In some embodiments, image source 602 can be any suitable source of image data (e.g., measurement data, images reconstructed from measurement data), such as an magnetic resonance imaging system, another computing device (e.g., a server storing image data), and so on. In some embodiments, image source 602 can be local to computing device 650. For example, image source 602 can be incorporated with computing device 650 (e.g., computing device 650 can be configured as part of a device for capturing, scanning, and/or storing images). As another example, image source 602 can be connected to computing device 650 by a cable, a direct wireless link, and so on. Additionally or alternatively, in some embodiments, image source 602 can be located locally and/or remotely from computing device 650, and can communicate data to computing device 650 (and/or server 652) via a communication network (e.g., communication network 654).

In some embodiments, communication network 654 can be any suitable communication network or combination of communication networks. For example, communication network 654 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, and so on. In some embodiments, communication network 654 can be a local area network, a wide area network, a public network (e.g., the Internet), a private or semi-private network (e.g., a corporate or university intranet), any other suitable type of network, or any suitable combination of networks. Communications links can each be any suitable communications link or combination of communications links, such as wired links, fiber optic links, Wi-Fi links, Bluetooth links, cellular links, and so on.

Referring now to FIG. 6B, an example of hardware 700 that can be used to implement image source 602, computing device 650, and server 652 in accordance with some embodiments of the systems and methods described in the present disclosure is shown. As shown in FIG. 6B, in some embodiments, computing device 650 can include a processor 702, a display 704, one or more inputs 706, one or more communication systems 708, and/or memory 710. In some embodiments, processor 702 can be any suitable hardware processor or combination of processors, such as a central processing unit (“CPU”), a graphics processing unit (“GPU”), and so on. In some embodiments, display 704 can include any suitable display devices, such as a computer monitor, a touchscreen, a television, and so on. In some embodiments, inputs 706 can include any suitable input devices and/or sensors that can be used to receive user input, such as a keyboard, a mouse, a touchscreen, a microphone, and so on.

In some embodiments, communications systems 708 can include any suitable hardware, firmware, and/or software for communicating information over communication network 654 and/or any other suitable communication networks. For example, communications systems 708 can include one or more transceivers, one or more communication chips and/or chip sets, and so on. In a more particular example, communications systems 708 can include hardware, firmware and/or software that can be used to establish a Wi-Fi connection, a Bluetooth connection, a cellular connection, an Ethernet connection, and so on.

In some embodiments, memory 710 can include any suitable storage device or devices that can be used to store instructions, values, data, or the like, that can be used, for example, by processor 702 to present content using display 704, to communicate with server 652 via communications system(s) 708, and so on. Memory 710 can include any suitable volatile memory, non-volatile memory, storage, or any suitable combination thereof. For example, memory 710 can include RAM, ROM, EEPROM, one or more flash drives, one or more hard disks, one or more solid state drives, one or more optical drives, and so on. In some embodiments, memory 710 can have encoded thereon, or otherwise stored therein, a computer program for controlling operation of computing device 650. In such embodiments, processor 702 can execute at least a portion of the computer program to present content (e.g., images, user interfaces, graphics, tables), receive content from server 652, transmit information to server 652, and so on.

In some embodiments, server 652 can include a processor 712, a display 714, one or more inputs 716, one or more communications systems 718, and/or memory 720. In some embodiments, processor 712 can be any suitable hardware processor or combination of processors, such as a CPU, a GPU, and so on. In some embodiments, display 714 can include any suitable display devices, such as a computer monitor, a touchscreen, a television, and so on. In some embodiments, inputs 716 can include any suitable input devices and/or sensors that can be used to receive user input, such as a keyboard, a mouse, a touchscreen, a microphone, and so on.

In some embodiments, communications systems 718 can include any suitable hardware, firmware, and/or software for communicating information over communication network 654 and/or any other suitable communication networks. For example, communications systems 718 can include one or more transceivers, one or more communication chips and/or chip sets, and so on. In a more particular example, communications systems 718 can include hardware, firmware and/or software that can be used to establish a Wi-Fi connection, a Bluetooth connection, a cellular connection, an Ethernet connection, and so on.

In some embodiments, memory 720 can include any suitable storage device or devices that can be used to store instructions, values, data, or the like, that can be used, for example, by processor 712 to present content using display 714, to communicate with one or more computing devices 650, and so on. Memory 720 can include any suitable volatile memory, non-volatile memory, storage, or any suitable combination thereof. For example, memory 720 can include RAM, ROM, EEPROM, one or more flash drives, one or more hard disks, one or more solid state drives, one or more optical drives, and so on. In some embodiments, memory 720 can have encoded thereon a server program for controlling operation of server 652. In such embodiments, processor 712 can execute at least a portion of the server program to transmit information and/or content (e.g., data, images, a user interface) to one or more computing devices 650, receive information and/or content from one or more computing devices 650, receive instructions from one or more devices (e.g., a personal computer, a laptop computer, a tablet computer, a smartphone), and so on.

In some embodiments, image source 602 can include a processor 722, one or more image acquisition systems 724, one or more communications systems 726, and/or memory 728. In some embodiments, processor 722 can be any suitable hardware processor or combination of processors, such as a CPU, a GPU, and so on. In some embodiments, the one or more image acquisition systems 724 are generally configured to acquire data, images, or both, and can include an MR imaging system. Additionally or alternatively, in some embodiments, one or more image acquisition systems 724 can include any suitable hardware, firmware, and/or software for coupling to and/or controlling operations of an MR imaging system. In some embodiments, one or more portions of the one or more image acquisition systems 724 can be removable and/or replaceable.

Note that, although not shown, image source 602 can include any suitable inputs and/or outputs. For example, image source 602 can include input devices and/or sensors that can be used to receive user input, such as a keyboard, a mouse, a touchscreen, a microphone, a trackpad, a trackball, and so on. As another example, image source 602 can include any suitable display devices, such as a computer monitor, a touchscreen, a television, etc., one or more speakers, and so on.

In some embodiments, communications systems 726 can include any suitable hardware, firmware, and/or software for communicating information to computing device 650 (and, in some embodiments, over communication network 654 and/or any other suitable communication networks). For example, communications systems 726 can include one or more transceivers, one or more communication chips and/or chip sets, and so on. In a more particular example, communications systems 726 can include hardware, firmware and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 728 can include any suitable storage device or devices that can be used to store instructions, values, data, or the like, that can be used, for example, by processor 722 to control the one or more image acquisition systems 724, and/or receive data from the one or more image acquisition systems 724; to images from data; present content (e.g., images, a user interface) using a display; communicate with one or more computing devices 650; and so on. Memory 728 can include any suitable volatile memory, non-volatile memory, storage, or any suitable combination thereof. For example, memory 728 can include RAM, ROM, EEPROM, one or more flash drives, one or more hard disks, one or more solid state drives, one or more optical drives, and so on. In some embodiments, memory 728 can have encoded thereon, or otherwise stored therein, a program for controlling operation of image source 602. In such embodiments, processor 722 can execute at least a portion of the program to generate images, transmit information and/or content (e.g., data, images) to one or more computing devices 650, receive information and/or content from one or more computing devices 650, receive instructions from one or more devices (e.g., a personal computer, a laptop computer, a tablet computer, a smartphone, etc.), and so on.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A system for performing a resting-state functional magnetic resonance image (rs-fMRI) reconstruction of functional magnetic resonance imaging (fMRI) dataset, comprising:
a magnet system configured to generate a polarizing magnetic field about at least a portion of a subject;
a magnetic gradient system including a plurality of magnetic gradient coils configured to apply at least one magnetic gradient field to the polarizing magnetic field;
a radio frequency (RF) system configured to apply an RF field to the subject and to receive magnetic resonance signals from the subject using a coil array;
a computer system programmed to:
control the magnetic gradient system and the RF system to an acquire fMRI dataset using at least one of a task-based fMRI data acquisition or an rs-fMRI data acquisition;
during the at least one of the task-based fMRI data acquisition or the rs-fMRI data acquisition, reconstruct the fMRI dataset using an rs-fMRI reconstruction process to generate at least one resting-state (rs) image;
during the at least one of the task-based fMRI data acquisition or the rs-fMRI data acquisition, compare the at least one rs image to a reference image to determine motion of the subject during the at least one of the task-based fMRI data acquisition or the rs-fMRI data acquisition;
determine a displacement of the subject corresponding to the motion of the subject;
during the at least one of the task-based fMRI data acquisition or the rs-fMRI data acquisition, generate at least one of an alert or a real-time indication of the displacement that is communicated to an operator of the MRI system.

2. The system of claim 1, wherein the computer system is further programmed to indicate at least one of a quantity of the fMRI dataset affected by the displacement or a portion of the at least one of the task-based fMRI data acquisition or the rs-fMRI data acquisition affected by the displacement.

3. The system of claim 1, wherein the computer system is further programmed to determine the motion of the subject using six alignment parameters, wherein the six alignment parameters are x, y, z, θ_{χ}, θ_{y}, and θ_{ζ}.

4. The system of claim 1, wherein the reference image includes a preceding rs-image reconstructed from the fMRI dataset using the rs-fMRI reconstruction process.

5. The system of claim 1, wherein the computer system is further programmed to predict a quantity of the fMRI dataset that is below a predetermined threshold for displacement and further comprising a display configured to display the predicted quantity of fMRI dataset below the threshold in real time as the subject during the at least one of the task-based fMRI data acquisition or the rs-fMRI data acquisition.

6. The system of claim 5, wherein predicting the quantity of fMRI dataset below the threshold includes applying a linear model (y=mx+b), wherein y is a predicted quantity of the fMRI dataset below the threshold available upon completion of during the at least one of the task-based fMRI data acquisition or the rs-fMRI data acquisition, x is a count of a consecutive dataset, and m and b are estimations for each subject in real time.

7. The system of claim 1, wherein comparing the at least one rs-fMRI image to the reference image comprises calculating a series of rigid body transforms, T_{;}, wherein i indexes the spatial registration of the at least one rs-fMRI image to at least one preceding image reconstructed form the fMRI dataset, wherein each of the series of rigid body transforms is calculated by minimizing a registration error:
εᵢ= [symbol] (sIi(T(x))−I₁(\vec{x}))² [symbol]
where I(x) represents an image intensity at locus x and s represents a scalar factor that compensates for fluctuations in mean signal intensity.

8. The system of claim 7, wherein each of the series of rigid body transforms is represented by a combination of rotations and displacements given by:
$${T}_{i} = [ \begin{matrix}{R}_{i} & {d}_{i} \\ 0 & 1\end{matrix} ]$$
wherein Rᵢ represents a 3×3 matrix of rotations, d; represents a 3×1 column vector of displacements, and wherein Rᵢ represents three elementary rotations at each axes.

9. The system of claim 1, wherein determining total displacement includes subtracting displacement for a preceding one of the at least one rs-fMRI image from a displacement for a current image of the at least one rs-fMRI image.

10. The system of claim 1, further comprising a sensory feedback system configured to deliver sensory feedback to the subject based on the displacement to prompt mitigation of the displacement or potential future displacement.

11. A computer-implemented method for resting-state functional magnetic resonance imaging (rs-fMRI) reconstruction of functional magnetic resonance imaging (fMRI) dataset, the computer-implemented method comprising:
receiving, using a computing device that includes at least one processor in communication with at least one memory device and that is in communication with a magnetic resonance imaging (MRI) system, an fMRI dataset from the MRI system while the MRI system is performing at least one of a task-based fMRI data acquisition or a rs-fMRI data acquisition;
performing an rs-fMRI reconstructing of the fMRI dataset, using the computing device, to generate rs-fMRI images;
comparing, using the computing device and during the at least one of a task-based fMRI data acquisition or the rs-fMRI data acquisition, the rs-fMRI image to at least one reference image;
determining, using the computing device, motion of the subject using based on comparing the rs-fMRI image to the at least one reference image; and
communicating, using the computing device, an alert to an operator of the MRI system indicating motion detected during the at least one of the task-based fMRI data acquisition or the rs-fMRI data acquisition.

12. The computer-implemented method of claim 11, wherein determining the motion of the subject includes using six alignment parameters, wherein the six alignment parameters are x, y, z, θ_{χ}, θ_{y}, and θ_{ζ}.

13. The computer-implemented method of claim 12, wherein the six alignment parameters include at least one of frame-wise or slice-wise alignment.

14. The computer-implemented method of claim 11, wherein the reference dataset includes a preceding image to the rs-fMRI image.

15. The computer-implemented method of claim 11, further comprising:
predicting, using the computing device, a quantity of fMRI dataset that is below a predetermined threshold for displacement; and
communicating, using the computing device, the predicted quantity of fMRI dataset below the threshold in real time during the at least one of the task-based fMRI data acquisition or the rs-fMRI data acquisition.

16. The computer-implemented method of claim 15, wherein predicting the quantity of fMRI dataset below the threshold includes applying a linear model (y=mx+b),
wherein y is a predicted quantity of fMRI dataset below the threshold available upon completion of the at least one of the task-based fMRI data acquisition or the rs-fMRI data acquisition, x is a count of consecutive dataset, and m and b are estimations for each subject in real time.

17. The computer-implemented method of claim 11, wherein comparing the rs-fMRI image to at least one reference image comprises calculating a series of rigid body transforms, T_{;}, wherein i indexes the spatial registration of the rs-fMRI image to the at least one reference image corresponding to a preceding portion image to the rs-fMRI image, wherein each of the series of rigid body transforms is calculated by minimizing a registration error:
εᵢ= [symbol] (sIi(T(x))−I₁(\vec{x}))² [symbol]
where I(x) represents an image intensity at locus x and s represents a scalar factor that compensates for fluctuations in mean signal intensity.

18. The computer-implemented method of claim 17, wherein each of the series of rigid body transforms is represented by a combination of rotations and displacements given by:
$${T}_{i} = [ \begin{matrix}{R}_{i} & {d}_{i} \\ 0 & 1\end{matrix} ]$$
wherein Rᵢ represents a 3×3 matrix of rotations, d; represents a 3×1 column vector of displacements, and wherein Rᵢ represents three elementary rotations at each axes.

19. The computer-implemented method of claim 11, wherein determining, the total displacement includes subtracting displacement for a preceding image to the rs-fMRI image from a displacement for the rs-fMRI image.

20. The computer-implemented method of claim 11, further comprising, using the computing device, delivering sensory feedback to the subject based on the displacement to prompt mitigation of the displacement or potential future displacement.

21. A system for generating resting-state functional magnetic resonance images (rs-fMRI) comprising:
a magnet system configured to generate a polarizing magnetic field about at least a portion of a subject;
a magnetic gradient system including a plurality of magnetic gradient coils configured to apply at least one magnetic gradient field to the polarizing magnetic field;
a radio frequency (RF) system configured to apply an RF field to the subject and to receive magnetic resonance signals from the subject using a coil array;
a computer system programmed to:
control the gradient system and the RF system to perform a task-based fMRI acquisition to acquire task-based fMRI dataset from the subject;
reconstruct the task-based fMRI dataset using an rs-fMRI reconstruction process to generate rs-fMRI images from the task-based fMRI dataset.

22. A system for performing resting-state functional magnetic resonance imaging (rs-fMRI) comprising:
a magnet system configured to generate a polarizing magnetic field about at least a portion of a subject;
a magnetic gradient system including a plurality of magnetic gradient coils configured to apply at least one magnetic gradient field to the polarizing magnetic field;
a radio frequency (RF) system configured to apply an RF field to the subject and to receive magnetic resonance signals from the subject using a coil array to form an rs-fMRI dataset according to an fMRI data acquisition;
a computer system programmed to:
receive the rs-fMRI dataset and compare the rs-fMRI dataset to reference dataset to determine motion of the subject;
determine a displacement of the subject corresponding to the motion of the subject; and
generate at least one of an alert or a real-time indication of the displacement that is communicated to an operator of the MRI system during the fMRI data acquisition.

23. A method for producing resting-state functional magnetic resonance imaging (rs-fMRI) images, the method comprising:
receiving functional magnetic resonance imaging (fMRI) data acquired from a subject as the subject is subjected to at least one of performing a task or experiencing a stimulus;
reconstructing the fMRI data acquired as the subject is subjected to at least one of performing a task or experiencing a stimulus using a resting-state fMRI (rs-fMRI) reconstruction process without accounting for the at least one of performing the task or experiencing the stimulus to generating rs-fMRI images; and
displaying the rs-fMRI images.
